(19) United States
(12) Patent Application Publication
(10) Pub. No.: US 2026/0114799 A1
(43) Pub. Date: Apr. 30, 2026

(54) PRESSURE INJURY ASSESSMENT

(71) Applicant: Hill-Rom Services, Inc., Batesville, IN (US)

(72) Inventors: Unnati Ojha, Apex, NC (US); Madhulika Chikkerur, Chicago, IL (US); Frederick Collin Davidson, Apex, NC (US); Jessica Lynn Jones Arnold, Greenville, SC (US); Cheng-En Lai, Chicago, IL (US); Melanie Tinney, Tucson, AZ (US)

(21) Appl. No.: 19/363,892

(22) Filed: Oct. 21, 2025

Related U.S. Application Data
(60) Provisional application No. 63/711,321, filed on Oct. 24, 2024.

(51) Int. Cl. A61B 5/00; A61G 7/057; G16H 10/20; G16H 40/67

(52) U.S. Cl. CPC A61B 5/447; A61B 5/4836; A61B 5/6892; A61B 5/7246; A61B 5/7275; A61G 7/05769; G16H 10/20; G16H 40/67; A61G 2203/36; A61G 2203/44

(57) ABSTRACT

A system for assessing pressure injuries in a healthcare facility. The system creates patient metadata for a patient and associates the patient metadata with medical equipment including a patient support apparatus having a controller that performs pressure injury prevention functions and a surface positioned on a frame of the patient support apparatus. The system records usage data from the medical equipment via a network interface. The usage data including duration and frequency measurements of pressure injury prevention functions. The system receives sensor data from sensors positioned on the patient support apparatus. The sensor data includes weight distribution and patient movement measurements. The system pairs the usage data and the sensor data to the patient metadata to create survey data that correlates patient risk factors with therapeutic interventions performed by the medical equipment and stores the survey data in a database accessible for automated completion of a survey.

### CROSS-REFERENCE TO RELATED APPLICATION

This application claims the benefit of U.S. Provisional Application No. 63/711,321, filed Oct. 24, 2024, the disclosure of which is hereby incorporated by reference in its entirety.

### BACKGROUND

The International Pressure Ulcer/Injury Prevalence (IPUP/IPIP) survey is a global initiative aimed at assessing the prevalence of pressure ulcers and injuries across various healthcare settings. The survey gathers data from hospitals, nursing homes, and community care facilities, providing valuable insights into the incidence and management of these injuries.

Key objectives of the IPUP/IPIP survey include identifying trends in prevalence rates, evaluating the effectiveness of prevention strategies, and fostering international collaboration in pressure ulcer care. Participants collect standardized data on patient demographics, the presence of pressure ulcers or injuries, and associated risk factors.

Findings from the IPUP/IPIP survey inform healthcare policies, enhance clinical practices, and improve patient outcomes by highlighting areas for targeted interventions. By providing a comprehensive overview of the current state of pressure ulcer prevalence and prevention, the survey supports healthcare providers, policymakers, and researchers in their efforts to reduce the occurrence of these injuries and enhance patient outcomes.

### SUMMARY

In general terms, the present disclosure relates to assessing prevalence of pressure injuries in a healthcare facility. In one possible configuration, a technical effect is provided by pairing usage data of medical equipment to patient metadata to create survey data that automates completion of a survey related to prevalence of pressure injuries in the healthcare facility while also enhancing accuracy and specificity of the survey. Various aspects are described in this disclosure, which include, but are not limited to, the following aspects.

One aspect relates to a system for assessing pressure injuries in a healthcare facility, the system comprising: at least one processing device; and a memory device storing instructions which, when executed by the at least one processing device, cause the at least one processing device to: create patient metadata for a patient; associate the patient metadata with medical equipment including a patient support apparatus having a controller configured to perform pressure injury prevention functions and a surface positioned on a frame of the patient support apparatus; record usage data from the medical equipment via a network interface, the usage data including duration and frequency measurements of pressure injury prevention functions including a surface mode function that controls inflation of the surface, a turn assist function that inflates zones of the surface to turn the patient left or right, and a rotation function that provides continuous lateral rotation; receive sensor data from one or more sensors positioned on the patient support apparatus, the sensor data including at least weight distribution and patient movement measurements; pair the usage data and the sensor data to the patient metadata to create survey data that correlates patient risk factors with therapeutic interventions performed by the medical equipment; and store the survey data in a database that is accessible for automated completion of a survey that assesses the prevalence of the pressure injuries in the healthcare facility.

Another aspect relates to a method of mitigating pressure injuries within a healthcare facility, the method comprising: creating patient metadata for a patient; associating the patient metadata with medical equipment including a patient support apparatus having a controller configured to perform pressure injury prevention functions and a surface positioned on a frame of the patient support apparatus; recording usage data from the medical equipment via a network interface, the usage data including duration and frequency measurements of pressure injury prevention functions including a surface mode function that controls inflation of the surface, a turn assist function that inflates zones of the surface to turn the patient left or right, and a rotation function that provides continuous lateral rotation; receiving sensor data from one or more sensors positioned on the patient support apparatus, the sensor data including at least weight distribution and patient movement measurements; pairing the usage data and the sensor data to the patient metadata to create survey data that correlates patient risk factors with actual therapeutic interventions performed by the medical equipment; and storing the survey data in a database that is accessible for automated completion of a survey that assesses the prevalence of the pressure injuries in the healthcare facility.

Another aspect relates to patient support apparatus for preventing pressure injuries in a healthcare facility, the patient support apparatus comprising: a frame configured to support a surface; a controller including a processing device and a memory device storing instructions which, when executed by the processing device, cause the processing device to: perform one or more functions related to pressure injury prevention, the one or more functions including a surface mode function, a turn assist function, and a rotation function; record usage data of the one or more functions related to pressure injury prevention; and transmit the usage data to a server for pairing with patient metadata to create survey data for assessing prevalence of pressure injuries in the healthcare facility; a network interface configured to communicate with the server; and a display panel configured to display user interfaces for controlling the one or more functions related to pressure injury prevention.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for assessing prevalence of pressure injuries in a healthcare facility.

FIG. 2 is an isometric view of an example of a patient support apparatus and a surface in the system of FIG. 1.

FIG. 3 schematically illustrates examples of a connectivity server and the patient support apparatus in the system of FIG. 1.

FIG. 4 schematically illustrates an example of a method for completing a survey of pressure injuries within the healthcare facility, the method can be performed by a survey application installed on the connectivity server of FIG. 3.

FIG. 5 illustrates an example of patient metadata that can be created by an operation of the method of FIG. 4.

FIG. 6 illustrates an example of a user interface displayed on a display panel of the patient support apparatus of FIG. 2.

FIG. 7 illustrates another example of a user interface displayed on the display panel of the patient support apparatus of FIG. 2.

FIG. 8 illustrates another example of a user interface displayed on the display panel of the patient support apparatus of FIG. 2.

FIG. 9 illustrates another example of a user interface displayed on the display panel of the patient support apparatus of FIG. 2.

### DETAILED DESCRIPTION

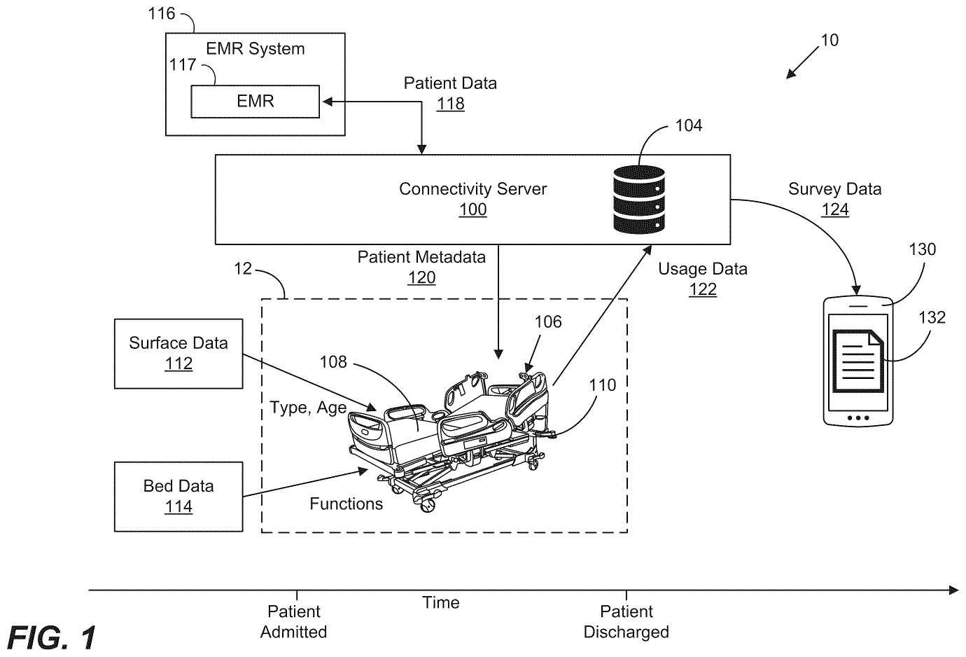

FIG. 1 schematically illustrates an example of a system 10 for assessing prevalence of pressure injuries in a healthcare facility. The system 10 includes a connectivity server 100 that maintains a database 104 for storage of survey data 124 accessible by a workstation device 130. The workstation device 130 can pull the survey data 124 from the database 104 to complete a survey 132 that assesses prevalence of pressure injuries in the healthcare facility. In some examples, the survey 132 is the International Pressure Ulcer/Injury Prevalence (IPUP/IPIP) survey. As will be described in more detail, the system 10 enhances the accuracy and specificity of the IPUP/IPIP survey, while also reducing the time required to complete the survey.

Illustrative examples of the healthcare facilities where the system 10 can be implemented include hospitals, long-term care facilities, nursing homes, and other types of healthcare facilities where pressure injuries may occur. A pressure injury, also known as a pressure ulcer, a bed sore, or a pressure sore, is damage to the skin and underlying tissue caused by prolonged or severe pressure. Pressure injuries can occur when an area of the body is in constant contact with an external surface, such as when a person is confined to a bed or a chair for a prolonged period of time and the person does not change positions regularly.

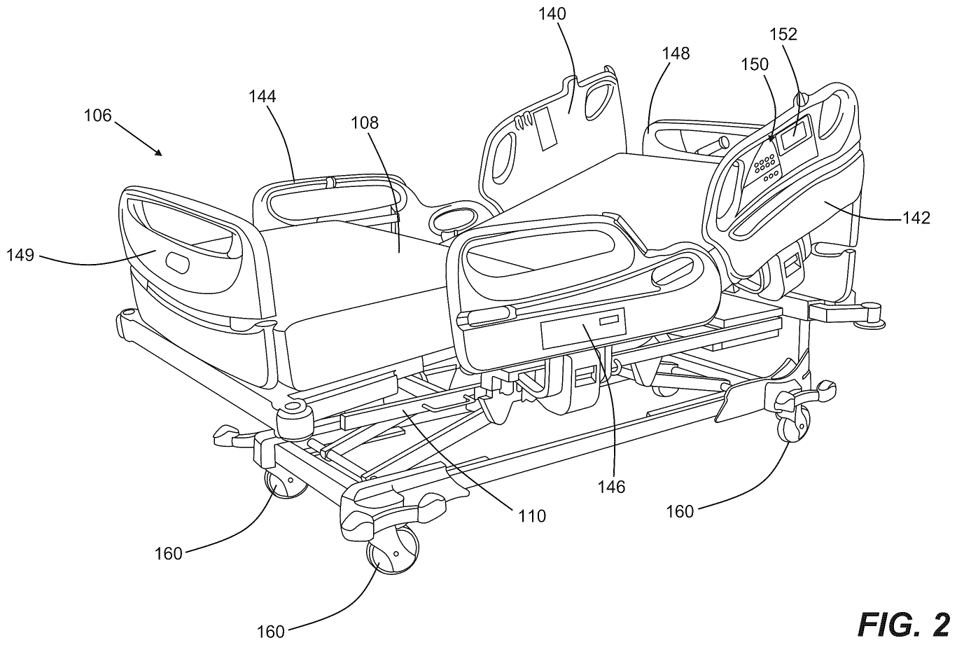

In the example illustrated in FIG. 1, the medical equipment inside the patient care environment 12 includes a patient support apparatus 106 and a surface 108 on the patient support apparatus 106. Illustrative examples of the patient support apparatus 106 can include a hospital bed, or similar types of apparatuses where the patient P rests for prolonged periods of time while admitted in the healthcare facility. Illustrative examples of the surface 108 can include a mattress that can be positioned onto a frame 110 of the patient support apparatus 106. The patient support apparatus 106 and the surface 108 are both described in more detail with reference to FIG. 2.

As shown in FIG. 1, the patient support apparatus 106 receives surface data 112 associated with the surface 108 that is positioned on the frame 110 of the patient support apparatus 106. The patient support apparatus 106 can store the surface data 112 onto a memory for reference and further processing.

An illustrative example of the surface data 112 can include an identification (ID) number associated with a particular unit, type, or model of the surface 108. The surface data 112 can further include an age of the surface 108 that can be determined from a duration and/or a frequency of usage. The duration of usage can include a total time measurement of how long a plurality of patients have been resting on the surface 108 during the lifetime of the surface 108. The frequency of usage can include how many times the surface 108 has been used by different patients admitted to the healthcare facility. As an illustrative example, surfaces 108 in the healthcare facility that have been used for longer durations of time and/or that have been more frequently used are considered older than surfaces 108 that have been used for shorter durations of time and/or that have been less frequently used by patients admitted to the healthcare facility.

As further shown in FIG. 1, the patient support apparatus 106 receives bed data 114. The patient support apparatus 106 can store the bed data 114 onto the memory for reference and further processing. The bed data 114 can include an identification of one or more functions that are available on the patient support apparatus 106 to prevent or mitigate pressure injuries. The identification of the one or more functions that are available to prevent pressure injuries can be based on an ID number associated with a unit, type, or model of the patient support apparatus 106 and/or the ID number associated with the particular type or model of the surface 108.

The connectivity server 100 collects patient data 118 from an electronic medical record (EMR) 117 maintained by an EMR system 116. The EMR 117 belongs to a particular patient assigned to the patient support apparatus 106 and the surface 108. The patient data 118 can include patient demographic information such as patient identification (ID) number as well as the age, sex, height, weight, comorbidities, and diagnosis related group (DRG) of the patient.

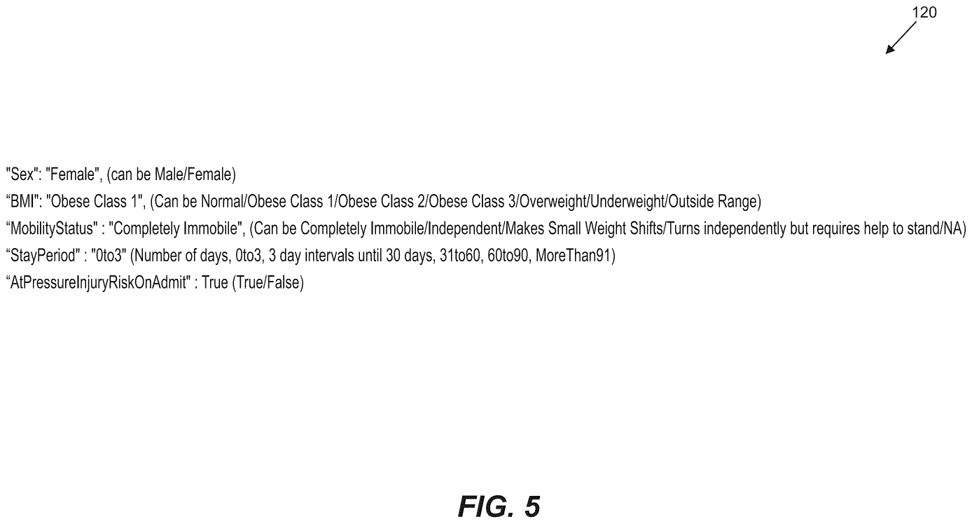

As will be described in more detail further below, the connectivity server 100 creates patient metadata 120 based on the patient data 118 collected from the EMR 117 of the EMR system 116. The patient metadata 120 includes condensed information relevant to pressure injury risk. The patient metadata 120 can include a format such as the one shown in FIG. 5.

The connectivity server 100 transmits patient metadata 120 to the patient support apparatus 106. The patient support apparatus 106 can store the patient metadata 120 onto the memory. A controller of the patient support apparatus 106 can use the patient metadata 120 to adjust control of one or more functions available on the patient support apparatus 106 to prevent or mitigate pressure injuries. The controller of the patient support apparatus 106 can also control a display on the patient support apparatus 106 or elsewhere based on the patient metadata 120.

Further, the connectivity server 100 receives usage data 122 from the patient support apparatus 106. The usage data 122 can include usage statistics of the one or more functions that are related to pressure injury prevention that are performed on the patient support apparatus 106 while the patient is admitted to the healthcare facility. For example, the usage data 122 can include a duration and frequency of usage of the one or more functions related to pressure injury prevention. The duration can include a time measurement of how long each of the one or more functions have been performed while a particular patient is admitted to the patient support apparatus 106. The frequency can include a quantity of how many times each of the one or more functions have been performed while the particular patient is admitted to the patient support apparatus 106. The one or more functions related to pressure injury prevention include a surface mode function, a turn assist function, and a rotation function that can be performed on the patient support apparatus 106. These types of functions will be described in more detail further below.

The usage data 122 can further include a duration and frequency of usage of the patient support apparatus 106 and the surface 108 by the particular patient. For example, the duration can include a time measurement of how long the particular patient has been resting on the surface 108. In further examples, the frequency of usage can include how many times the patient has left and returned to the surface 108 on the patient support apparatus 106.

As further shown in FIG. 1, the connectivity server 100 generates the survey data 124 based on the patient metadata 120 and the usage data 122 acquired from the one or more pieces of medical equipment inside the patient care environment 12. The survey data 124 is accessible on the database 104 by the workstation device 130. Examples of the workstation device 130 can include a desktop computer, a laptop computer, a tablet, a smartphone, and the like.

The workstation device 130 uses the survey data 124 to complete a survey 132 that assesses the prevalence of the pressure injuries in the healthcare facility. The workstation device 130 can automatically complete the survey 132 using the survey data 124. The survey 132 can include the International Pressure Ulcer/Injury Prevalence (IPUP/IPIP) survey.

In the example illustrated in FIG. 1, the surface data 112, the bed data 114, the patient data 118, the patient metadata 120, the usage data 122, and the survey data 124 are arranged along a timeline from when a patient is admitted to the healthcare facility to when the patient is discharged from the healthcare facility. For example, the surface data 112 and the bed data 114 can be acquired by the controller of the patient support apparatus 106 before the patient has been admitted since these pieces of data exist in the healthcare facility prior to the patient's admission.

After the patient is admitted to the healthcare facility, the connectivity server 100 receives the patient data 118 from the EMR system 116 and then creates the patient metadata 120. Accordingly, the controller of the patient support apparatus 106 acquires the patient metadata 120 after the patient is admitted to the healthcare facility.

After the patient is discharged from the healthcare facility, the connectivity server 100 collects the usage data 122 from the patient support apparatus 106. Then, the connectivity server 100 generates the survey data 124 which can be used by the workstation device 130 to complete the survey 132 after the patient is discharged from the healthcare facility.

FIG. 2 is an isometric view of an example of the patient support apparatus 106 and the surface 108. As shown in FIG. 2, the surface 108 is supported on the frame 110. Further, the frame 110 includes a plurality of siderails such as upper left and right siderails 140, 142, and lower left and right siderails 144, 146. The frame 110 can further include a headboard 148 and a footboard 149. The frame 110 is supported on a plurality of casters 160 such that the patient support apparatus 106 can be wheeled around the healthcare facility.

As shown in FIG. 2, the patient support apparatus 106 includes a control panel 150 and a display panel 152 on the upper right siderail 142. The control panel 150 can include a plurality of controls such as to adjust the articulation of one or more portions of the frame 110. The control panel 150 can further include controls to raise and lower a platform of the frame 110 that supports the surface 108 for adjusting the height of the surface 108 relative to the ground.

The display panel 152 can include a touchscreen display that provides additional controls such as to control one or more functions or therapies provided by the patient support apparatus 106. The display panel 152 can further display alerts generated by the controller of the patient support apparatus 106 based on measurements detected by one or more sensors on the frame 110 of the patient support apparatus 106 and/or included on the surface 108.

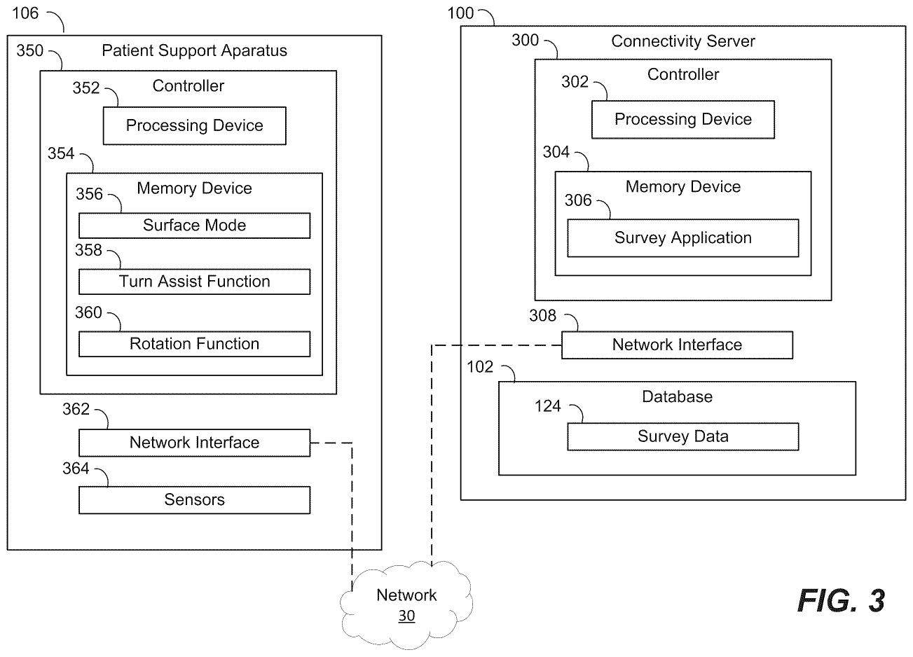

FIG. 3 schematically illustrates examples of the connectivity server 100 and the patient support apparatus 106. As shown in FIG. 3, the connectivity server 100 includes a controller 300 having a processing device 302 and a memory device 304. In some examples, the processing device 302 and the memory device 304 can be part of a processing circuitry that executes instructions causing the processing circuitry to perform aspects described herein.

The processing device 302 is an example of a processing unit such as a central processing unit (CPU). The processing device 302 can include one or more central processing units (CPUs). In some examples, the processing device 302 can include one or more digital signal processors, field-programmable gate arrays, or other electronic processing devices.

The memory device 304 stores a survey application 306 which generates the survey data 124 that can be accessed by one or more workstation devices 130 for completion of the survey 132. As shown in FIG. 3, the survey data 124 is stored in the database 104.

The memory device 304 operates to store data and instructions for execution by the processing device 302 of the controller 300. The memory device 304 includes computer-readable media, which may include any media that is accessible by the processing device 302. By way of illustrative example, the computer-readable media can include both computer-readable storage media and computer-readable communication media.

The computer-readable storage media includes volatile and nonvolatile, removable and non-removable media implemented in any device configured to store information such as computer-readable instructions, data structures, program modules, or other data. The computer-readable storage media can include, but is not limited to, random access memory, read only memory, electrically erasable programmable read only memory, flash memory, and other memory technology, including any medium that can be used to store information that can be accessed by the processing device 302. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are within the scope of computer-readable media.

As further shown in FIG. 3, the connectivity server 100 includes a network interface 308 that provides connection to a network 30. The network interface 308 can include wired interfaces and/or wireless interfaces. The connectivity server 100 can communicate with other systems and devices over the network 30. The network 30 can include wired connections, wireless connections, or any combinations thereof for establishing data communications.

In the example shown in FIG. 3, the connectivity server 100 communicates with the patient support apparatus 106 over the network 30. For example, the connectivity server 100 can transmit the patient metadata 120 to the patient support apparatus 106 over the network 30. Also, the connectivity server 100 can receive the usage data 122 from the patient support apparatus 106 over the network 30. Alternatively, the connectivity server 100 can transmit the patient metadata 120 directly to the patient support apparatus 106 and can receive the usage data 122 directly from the patient support apparatus 106. The communications between the connectivity server 100 and the patient support apparatus 106, whether accomplished directly or over the network 30, can use Substitutable Medical Applications and Reusable Technologies (SMART) on Fast Healthcare Interoperability Resources (FIHR), or other healthcare information protocols.

In some further examples, the connectivity server 100 receives over the network 30 the patient data 118 from the EMR system 116. Also, the workstation device 130 can pull the survey data 124 from the connectivity server 100 using the network 30.

As further shown in FIG. 3, the patient support apparatus 106 also includes a controller 350 having a processing device 352 and a memory device 354 that can share similar aspects with the controller 300, the processing device 302, and the memory device 304 of the connectivity server 100. The patient support apparatus 106 also includes a network interface 362 for connection to the network 30, which as described above, can facilitate communications between the patient support apparatus 106 and the connectivity server 100. The network interface 362 can share similar aspects with the network interface 308 of the connectivity server 100.

As further shown in FIG. 3, the memory device 354 of the controller 350 stores a surface mode function 356, a turn assist function 358, and a rotation function 360. The surface mode function 356, the turn assist function 358, and the rotation function 360 control aspects of the surface 108 to prevent or mitigate pressure injuries. The processing device 352 executes the surface mode function 356, the turn assist function 358, and the rotation function 360 such that the controller 350 performs these functions which are related to pressure injury prevention.

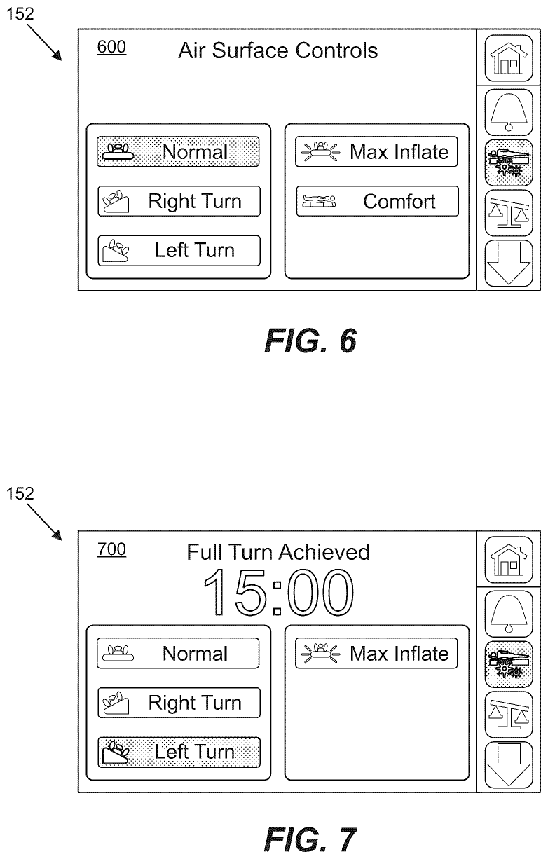

The surface mode function 356 controls the inflation of the surface 108 to operate between a normal mode, a max inflate mode, and a patient comfort mode. The normal mode, the max inflate mode, and the patient comfort mode can be selected on the display panel 152 on the patient support apparatus 106, as shown in the examples provided in FIGS. 6 and 7.

The normal mode of the surface 108 provides continuous full-body pressure redistribution for the patient. The surface 108 automatically adjusts to accommodate changes in weight distribution. The max inflate mode maximizes the firmness of the surface 108 such as to assist in patient mattress-to-mattress transfers and/or adjustment of the patient's position. The patient comfort mode allows adjustment of the pressure settings for one or more zones on the surface 108 to enhance patient comfort while still providing pressure redistribution.

The turn assist function 358 helps to turn the patient to the left or right. The turn assist function 358 includes a right turn control to turn the patient to the patient's right side and a left turn control to turn the patient to the patient's left side. The right and left turn controls are displayed on the display panel 152 on the upper right siderail 142 of the patient support apparatus 106, as shown in the examples provided in FIGS. 6 and 7.

The siderail that the patient is turning towards must be in the up position to activate the turn assist function 358. If the siderail is down, an alarm will sound, and a message will show on the display panel 152 to let the caregiver know that the siderail is lowered. Once the patient starts to turn, the siderail the patient is turning away from can be lowered for easier patient access. A beep will sound as a safety alert and a 15-minute countdown will show on the display panel 152 when the surface 108 reaches the full turn. The turn assist function 358 can be stopped during inflation or deflation by pressing a stop turn control on the display panel 152.

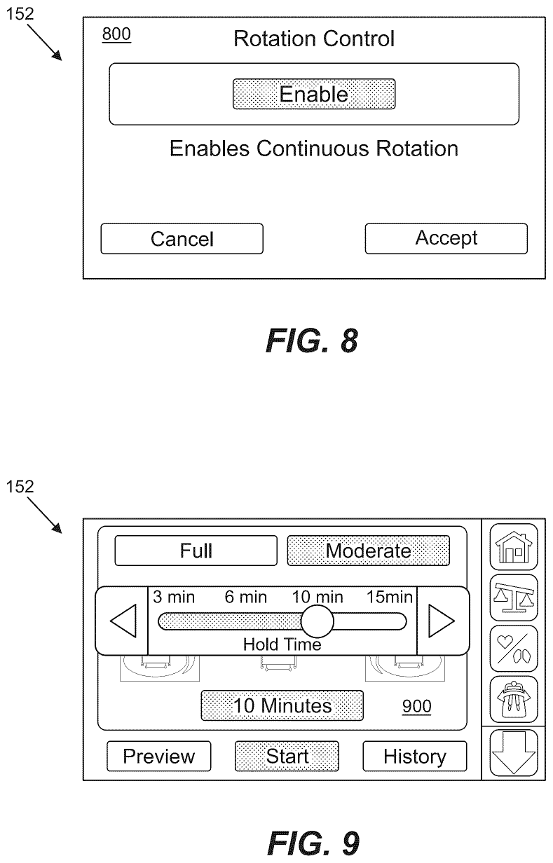

The rotation function 360 provides gentle, side-to-side, continuous lateral rotation (CLR) to help prevent or mitigate pressure injuries. Patients can be positioned laterally on the right or left side with varying amounts of turn and pause times to match each individual patient's condition. Pressure redistribution is provided when the rotation mode is active. One or more settings of the rotation function 360 can be adjusted using the display panel 152, as shown in the examples provided in FIGS. 8 and 9, which are described further below in more detail.

As further shown in FIG. 3, the patient support apparatus 106 further includes one or more sensors 364 such as load sensors that detect the weight distribution and movements of a patient while resting on the hospital bed. The load sensors can include one or more load cells, piezoelectric pressure sensors, strain gauges, and other types of sensors that can be positioned underneath the surface 108 to measure patient weight distribution and motion.

The one or more sensors 364 can further include a head section angle sensor that measures an angle of the head section of the frame 110, a lift system position sensor that measures a height of the frame 110, and one or more siderail position sensors that measure whether the siderails of the frame 110 are in the deployed position or in a stowed position.

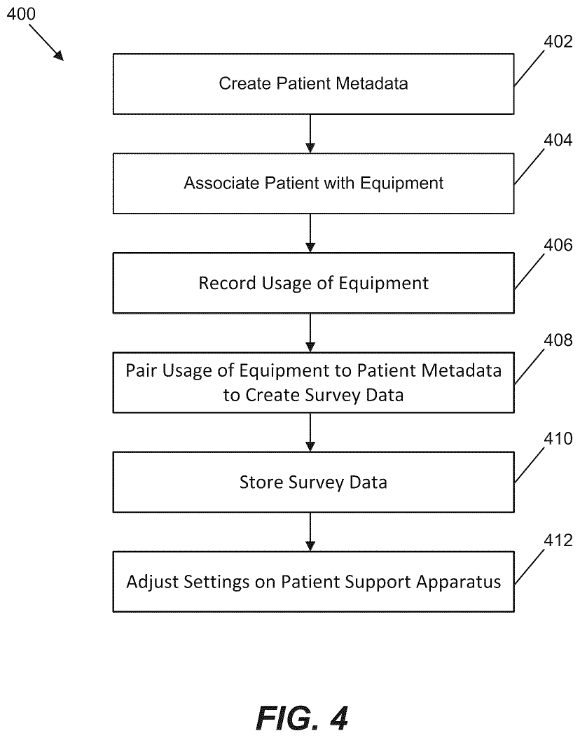

FIG. 4 schematically illustrates an example of a method 400 for completing a survey of pressure injuries within the healthcare facility. In some examples, the method 400 is performed by the survey application 306 installed on the memory device 304 of the connectivity server 100. The method 400 includes an operation 402 of creating the patient metadata 120 for a patient admitted to the patient support apparatus 106. Operation 402 can include creating the patient metadata 120 based on the patient data 118 received from the EMR 117. The patient metadata 120 can be structured to include classifiers (e.g., sex, BMI, mobility status, stay period, and pressure injury risk) that are organized into categories.

FIG. 5 illustrates an example of the patient metadata 120 that can be created by operation 402 of the method 400. As shown in FIG. 5, the patient metadata 120 is structured to include classifiers that are organized into categories. For example, the patient metadata 120 includes an identification (ID) number of the patient as well as demographic and physiological information such as a sex classifier that includes male or female categories and a body mass index (BMI) classifier that includes normal, obese class 1, obese class 2, obese class 3, overweight, underweight, and outside range categories. The patient metadata 120 can further include a mobility status classifier that includes completely immobile, independent, makes small weight shifts, turns independently but requires help to stand, and not available categories. The patient metadata 120 can further include a stay period classifier that includes 0 to 3 days, 4 to 7 days, 8 to 11 days, 12 to 15 days, 16 to 19 days, 20 to 23 days, 24 to 27 days, 28 to 30 days, 31 to 60 days, 60 to 90 days, and more than 91 days. The patient metadata 120 further includes a pressure injury risk upon admission classifier that includes true and false categories.

Referring back to FIG. 4, the method 400 includes an operation 404 of associating the patient metadata 120 created in operation 402 with medical equipment such as the patient support apparatus 106 and the surface 108. Operation 404 can include associating the ID number of the patient with the ID numbers of the patient support apparatus 106 and the surface 108. By associating the patient metadata 120 with the medical equipment, usage data related to the use of the medical equipment is associated with the patient metadata 120.

The method 400 includes an operation 406 of recording the usage data 122 (see FIG. 1) of the medical equipment. Operation 406 can include recording the usage data 122 of one or more functions related to pressure injury prevention such as the surface mode function 356, the turn assist function 358, the rotation function 360, and other functions performed on the patient support apparatus 106. Operation 406 can include recording a duration and/or a frequency of usage of the one or more functions related to pressure injury prevention. For example, operation 406 can include recording a time measurement (i.e., duration) and/or a quantity of times (i.e., frequency) of execution of each of the surface mode function 356, the turn assist function 358, and the rotation function 360 on the surface 108 while the patient was admitted to the patient support apparatus 106. In some examples, operation 406 can include recording an age of the surface 108 based on prior usage of the surface 108 in the healthcare facility.

The method 400 includes an operation 408 of pairing the usage data 122 of the medical equipment to the patient metadata 120 to create the survey data 124. Thus, the survey data 124 includes a pairing between the patient metadata 120 which includes classifiers (e.g., sex, BMI, mobility status, stay period, and pressure injury risk) that are organized into categories and the usage data 122 which includes metrics such as a duration time measurement and/or a frequency of usage of the one or more functions related to pressure injury prevention such as the surface mode function 356, the turn assist function 358, and the rotation function 360.

In some examples, operation 408 also includes pairing sensor data captured by the one or more sensors 364 to the patient metadata 120. The sensor data can include weight distribution and movement measurements of the patient detected by the one or more sensors 364 while resting on the surface 108. The sensor data can further include an angle of the head section of the frame 110, a height of the frame 110, and detection of whether the siderails of the frame 110 are in the deployed position or in the stowed position. The pairing or matching between the patient metadata 120, the usage data 122, and the sensor data creates a new dataset (i.e., the survey data 124) that allows automated completion of the survey 132 by the system 10.

The method 400 includes an operation 410 of storing the survey data 124 in the database 104 accessible by the workstation device 130. Upon completion of the survey 132, the prevalence of pressure injuries in the healthcare facility can be assessed. In some examples, the survey 132 is the International Pressure Ulcer/Injury Prevalence (IPUP/IPIP) survey.

In some instances, operation 410 includes automatically completing the survey 132 using the survey data 124. For example, the connectivity server 100 can automatically complete the survey 132 using the survey data 124. Alternatively, the workstation device 130 can pull the survey data 124 from the database 104 to automatically complete the survey 132.

Current methods of completion of the IPUP/IPIP survey typically require manual responses to a multitude of survey questions specific to the patient, the environment of care, and the equipment used by the patient. This is a tedious process that can require several hours to complete the survey. Furthermore, the data entered related to the age and usage of the equipment typically includes approximations. In view of the foregoing, the system 10 and the method 400 provide acquisition of more specific and detailed data about the usage of the equipment (e.g., the patient support apparatus 106 and the surface 108) that can aid in creating better reports from the IPUP/IPIP survey. Further, the system 10 and the method 400 automate completion of the IPIP/IPUP survey by directly feeding the usage data from the patient support apparatuses 106 and the surfaces 108 to the IPIP/IPUP survey. Advantageously, this can result in a reduction in the occurrence of pressure ulcers and injuries in the healthcare facility where the system 10 and the method 400 are implemented. Further, the system 10 and the method 400 can eliminate the time needed to manually enter the data for completion of the IPUP/IPIP survey.

In some examples, the method 400 can include an operation 412 of adjusting on the patient support apparatus 106 performance of the one or more functions related to pressure injury prevention based on the survey data 124. Operation 412 can include adjusting operation of any of the surface mode function 356, the turn assist function 358, and the rotation function 360.

FIG. 6 illustrates an example of a user interface 600 displayed on the display panel 152 of the patient support apparatus 106. In this example, the normal mode is selected as being activated on the patient support apparatus 106, while the max inflate mode and the patient comfort mode are not selected. In some examples, the controller 350 (see FIG. 3) of the patient support apparatus 106 prevents selection of one or more of the normal mode, the max inflate mode, and the patient comfort mode based on the survey data 124. Alternatively, the controller 350 can display an alert that recommends a selection of one or more of the normal mode, the max inflate mode, and the patient comfort mode based on the survey data 124.

FIG. 7 illustrates another example of a user interface 700 displayed on the display panel 152 of the patient support apparatus 106. In this example, the left turn control of the turn assist function 358 is shown as being selected. In some examples, the controller 350 of the patient support apparatus 106 displays an alert that reminds a caregiver based on the survey data 124 to select the left turn control and/or the right turn control of the turn assist function 358.

FIG. 8 illustrates another example of a user interface 800 displayed on the display panel 152 of the patient support apparatus 106. In this example, the user interface 800 allows a caregiver to accept an enablement of the rotation function 360 or to cancel. In some examples, the controller 350 of the patient support apparatus 106 automatically enables the rotation function 360 based on the survey data 124.

FIG. 9 illustrates another example of a user interface 900 displayed on the display panel 152 of the patient support apparatus 106. In this example, the user interface 900 displays controls for adjusting the settings of the rotation function 360 such as pause times (e.g., 3 minutes, 6 minutes, 10 minutes, and 15 minutes) and an intensity (e.g., full or moderate). In some examples, the controller 350 of the patient support apparatus 106 automatically adjusts the settings of the rotation function 360 based on the survey data 124.

The various embodiments described above are provided by way of illustration only and should not be construed to be limiting in any way. Various modifications can be made to the embodiments described above without departing from the true spirit and scope of the disclosure.

What is claimed is:

1. A system for assessing pressure injuries in a healthcare facility, the system comprising:
at least one processing device; and
a memory device storing instructions which, when executed by the at least one processing device, cause the at least one processing device to:
create patient metadata for a patient;
associate the patient metadata with medical equipment including a patient support apparatus having a controller configured to perform pressure injury prevention functions and a surface positioned on a frame of the patient support apparatus;
record usage data from the medical equipment via a network interface, the usage data including duration and frequency measurements of pressure injury prevention functions including a surface mode function that controls inflation of the surface, a turn assist function that inflates zones of the surface to turn the patient left or right, and a rotation function that provides continuous lateral rotation;
receive sensor data from one or more sensors positioned on the patient support apparatus, the sensor data including at least weight distribution and patient movement measurements;
pair the usage data and the sensor data to the patient metadata to create survey data that correlates patient risk factors with therapeutic interventions performed by the medical equipment; and
store the survey data in a database that is accessible for automated completion of a survey that assesses the prevalence of the pressure injuries in the healthcare facility.

2. The system of claim 1, wherein the surface mode function is configured to control inflation of the surface to operate between a normal mode that provides continuous full-body pressure redistribution, a max inflate mode that maximizes firmness of the surface to assist in patient transfers, and a patient comfort mode that allows adjustment of pressure settings for individual zones on the surface while maintaining pressure redistribution.

3. The system of claim 1, wherein the turn assist function includes a right turn control configured to turn the patient to the patient's right side and a left turn control configured to turn the patient to the patient's left side, wherein the turn assist function is configured to have a siderail on a side toward which the patient is turning to be in an up position before activating, and wherein the turn assist function includes safety features comprising an alarm and a countdown display when the surface reaches a full turn position.

4. The system of claim 1, wherein the rotation function provides continuous lateral rotation by positioning the patient laterally on a right side or a left side of the surface, wherein the rotation function includes adjustable settings including variable turn times and pause times that are individually configurable to match a patient's condition, and wherein the rotation function is configured to provide pressure redistribution while the rotation function is active.

5. The system of claim 1, wherein the usage data of the medical equipment in the patient care environment includes an age of the surface.

6. The system of claim 1, further comprising:
the patient support apparatus; and
the surface positioned on the frame of the patient support apparatus.

7. The system of claim 1, wherein the patient metadata is structured to include classifiers that are organized into categories.

8. The system of claim 1, wherein the memory device stores further instructions which, when executed by the at least one processing device, cause the at least one processing device to:
adjust on the patient support apparatus performance of the one or more functions related to pressure injury prevention based on the survey data.

9. The system of claim 1, wherein the memory device stores further instructions which, when executed by the at least one processing device, cause the at least one processing device to:
automatically complete the survey using the survey data.

10. The system of claim 1, wherein the survey is an International Pressure Ulcer/Injury Prevalence (IPUP/IPIP) survey.

11. A method of mitigating pressure injuries within a healthcare facility, the method comprising:
creating patient metadata for a patient;
associating the patient metadata with medical equipment including a patient support apparatus having a controller configured to perform pressure injury prevention functions and a surface positioned on a frame of the patient support apparatus;
recording usage data from the medical equipment via a network interface, the usage data including duration and frequency measurements of pressure injury prevention functions including a surface mode function that controls inflation of the surface, a turn assist function that inflates zones of the surface to turn the patient left or right, and a rotation function that provides continuous lateral rotation;
receiving sensor data from one or more sensors positioned on the patient support apparatus, the sensor data including at least weight distribution and patient movement measurements;
pairing the usage data and the sensor data to the patient metadata to create survey data that correlates patient risk factors with actual therapeutic interventions performed by the medical equipment; and
storing the survey data in a database that is accessible for automated completion of a survey that assesses the prevalence of the pressure injuries in the healthcare facility.

12. The method of claim 11, wherein the surface mode function includes controlling inflation of the surface to operate between a normal mode that provides continuous full-body pressure redistribution, a max inflate mode that maximizes firmness of the surface to assist in patient transfers, and a patient comfort mode that allows adjustment of pressure settings for individual zones on the surface while maintaining pressure redistribution.

13. The method of claim 11, wherein the turn assist function includes a right turn control configured to turn the patient to the patient's right side and a left turn control configured to turn the patient to the patient's left side, wherein the turn assist function is configured to have a siderail on a side toward which the patient is turning to be in an up position before activating, and wherein the turn assist function includes safety features comprising an alarm and a countdown display when the surface reaches a full turn position.

14. The method of claim 11, wherein the rotation function provides continuous lateral rotation by positioning the patient laterally on a right side or a left side of the surface, wherein the rotation function includes adjustable settings including variable turn times and pause times that are individually configurable to match a patient's condition, and wherein the rotation function is configured to provide pressure redistribution while the rotation function is active.

15. The method of claim 11, wherein the patient metadata is structured to include classifiers that are organized into categories.

16. The method of claim 11, further comprising:
adjusting on the patient support apparatus performance of the one or more functions related to pressure injury prevention based on the survey data.

17. The method of claim 11, further comprising:
automatically completing the survey using the survey data.

18. The method of claim 11, wherein the survey is an International Pressure Ulcer/Injury Prevalence (IPUP/IPIP) survey.

19. A patient support apparatus for preventing pressure injuries in a healthcare facility, the patient support apparatus comprising:
a frame configured to support a surface;
a controller including a processing device and a memory device storing instructions which, when executed by the processing device, cause the processing device to:
perform one or more functions related to pressure injury prevention, the one or more functions including a surface mode function, a turn assist function, and a rotation function;
record usage data of the one or more functions related to pressure injury prevention; and
transmit the usage data to a server for pairing with patient metadata to create survey data for assessing prevalence of pressure injuries in the healthcare facility;
a network interface configured to communicate with the server; and
a display panel configured to display user interfaces for controlling the one or more functions related to pressure injury prevention.

20. The patient support apparatus of claim 19, further comprising:
one or more sensors configured to detect at least one of weight distribution and movement of a patient on the surface, wherein the usage data includes sensor data from the one or more sensors.
